(19) United States
(12) Patent Application Publication
(10) Pub. No.: US 2008/0081375 A1
(43) Pub. Date: Apr. 3, 2008

(54) Metabolite detection using magnetic resonance

(75) Inventors: Yasvir A. Tesiram, Oklahoma City, OK (US); Rheal A. Towner, Piedmont, OK (US)

Correspondence Address: JOHN S. PRATT, ESQ;KILPATRICK STOCKTON, LLP, 1100 PEACHTREE STREET, ATLANTA, GA, 30309, US

(73) Assignee: Oklahoma Medical Research Foundation, Oklahoma City, OK (US)

(21) Appl. No.: 11/906,650

(22) Filed: Oct. 3, 2007

Related U.S. Application Data
(60) Provisional application No. 60/848,925, filed on Oct. 3, 2006.

(51) Int. Cl. G01N 23/00; C12Q 1/68

(52) U.S. Cl. 436/57; 435/6

(57) ABSTRACT

Methods using magnetic resonance, such as nuclear magnetic resonance (NMR) spectroscopy or magnetic resonance imaging (MRI), are provided for detecting metabolites in a sample. The methods are useful for the diagnosis or prognosis of a disease such as cancer and can also be used to determine or monitor a treatment protocol. The methods are useful in characterizing speciation in biological samples where mixtures are often encountered and chemical shifts of the same structural group of similar molecules can produce complicated overlapping resonances.

### CROSS REFERENCE TO RELATED APPLICATION

The present application claims priority to Provisional Patent Application No. 60/848,925 filed Oct. 3, 2006.

### FIELD OF THE INVENTION

The present application relates to methods for detecting metabolites using nuclear magnetic resonance (NMR) spectroscopy or magnetic resonance imaging (MRI). The methods are useful for determining alterations in metabolite levels and/or profiles in an individual for diagnosis, planning of physical or chemical intervention, and prognosis.

### BACKGROUND OF THE INVENTION

Studies in oncology have made it increasingly apparent that specific markers characterize tumor genesis. For example, choline phospholipid metabolism has been implicated in ovarian cancer (Iorio E., et al. Cancer Res. 2005; 65(20):9369-9376.), breast cancer (Whitehead T L., et al. Int. J. Oncology 2005; 27:257-263. and Katz-Bull R., Cancer Res. 2002; 62:1966-1970.), brain cancer (Klein J, et al. Neurochem. Int. 1993; 22(3):293-300.), and liver cancer (Kobliakov V. A., et al. Biochemistry 2001; 66:603-607).

Of these markers, the class of compound collectively known as lipids are often implicated as being altered. While numerous chemical species in the lipids class are present, some have specific structural signatures that are well known. For example, it has been shown that alterations in unsaturated fatty acyl groups of phospholipids exist in prostate tumors (Moore S., et al. J. Cancer 2005; 114:563-571 and Horrobin D. F., Am. J. Clin. Nutr. 1993; 57: 5732-5737) and breast cancer (Lane J, et al., Int. J. Mol. Med. 2003; 12: 253-257). The two fatty acid species oleic and linoleic each contain one and two double bonds, or “unsaturated” bonds (a vinyl moiety). However, these fatty acid are difficult to distinguish by NMR spectroscopy because the chemical environment of the vinyl groups in these two molecular species are similar. Even if two-dimensional (2D) spectra are collected over normal spectral widths (herein referred to as the “conventional NMR method”), the ability to distinguish these two fatty acids remains difficult because the adjacent bis-allyl nuclei are also chemically similar, thereby limiting resolution. Such limitations can be overcome to some extent by altering the electron density distribution of the molecule to produce chemical shifts with the use of chemical shift reagents such as lanthanide shift reagents. However, additional sample preparation steps are required, resulting in increased costs and prolonged time, and the administration of these reagents to patients poses health risks.

Another tumor marker being examined in cancer research is the signal in the NMR spectrum of the trimethyl group of choline, usually a side-chain of the phospholipid class. These phospholipid markers are often referred to collectively as “choline type” compounds. The trimethyl group of choline resonates at 3.2 ppm, but can be resolved in higher resolution spectra as originating from different compounds (‘chemical species’).

Although NMR and MRI technologies are being used for cancer research, currently available cancer detection methods using these technologies “lump together” overlapping resonances from classes of compounds and are unable to successfully detect individual chemical species. Therefore, what is needed is a detection method having the ability to distinguish between structural groups of similar molecules for accurate diagnosis, prognosis and treatment protocols.

### BRIEF SUMMARY OF THE INVENTION

The methods provided herein can be described as a collection of NMR methods using conventional NMR systems designed for either spectroscopy, spectroscopic imaging or the imaging of a patient or examination subject. The methods are useful for detecting known or uncharacterized pathological states using signals generated from metabolites. The methods utilize signal patterns, their amplitudes and area to determine the type and juncture of the disease state or disease states. Animal models of liver cancer are used in the examples below, but it will be understood by those skilled in the art that the methods are applicable to other cancers and other diseases.

In accordance with the methods provided herein, chemical species,

(i) are crudely, but quickly determined (referred to as the “screening method”),

(ii) are determined in a second method where unequivocal assignment of chemical species is made (“confirmatory method”) and,

(iii) spatial distribution is determined by a further refinement of the concepts of (i).

The methods are provided for detecting known or unknown metabolites using a nuclear magnetic resonance (NMR) spectrometer or a magnetic resonance imaging (MRI) instrument. The methods are useful for determining alterations in metabolite levels and/or profiles in a patient for diagnosis, planning of physical or chemical intervention, and prognosis. In one embodiment, the method is used to detect one or more metabolites in a sample obtained from a patient or examination subject. Samples include, but are not limited to, material excised (e.g. tissue biopsy), removed (e.g. blood, urine or saliva) or intact (e.g. whole organ), from or within a chosen region or regions of the examination subject. Metabolites are small endogenous molecules ranging in size to 2000 g/mole molecular weight. Detection of one or more metabolites indicates (diagnoses), and/or corroborates the existence of known pathological states, such as, for example, a type of cancer, by the detection of a single metabolite or number of metabolites.

In a first embodiment, the method allows the calculation of a characteristic measure for the rapid determination of the occurrence or non-occurrence of a persistent class of compound(s) and domination of one or more species within such a class via the analysis of a chosen signal or collection of signals from NMR induction decay or decays, or a subsequent processed induction signal represented as a spectrum or spectra, collected after the application of one or more RF pulses and delays, whether in the presence of static or pulsed field gradients. For example, in the simplest case, the area ratio of resonances at 2.8 ppm and 5.3 ppm in a proton NMR spectrum collected after the application of a single RF pulse can be used to determine the occurrence of one or more double bonds in the chemical species of the lipid class.

In a second embodiment, the method utilizes tailored RF pulses to determine directly or indirectly, the actual chemical species present in a class by limiting the NMR signal generated and thus detected to a set of resonances occurring in a very small region, or numerous small regions of the NMR spectrum and is a method of increasing NMR resolution. The information from these very high-resolution spectra may be used to determine the type and juncture of disease by simultaneously detecting one or more species within a class of compound by NMR.

In a third embodiment, spatial distribution maps of intact examination subjects are made by utilizing embodiments one and two above, to indicate the type and juncture of disease. Spatial distribution maps may be made using the methods previously described by Brown et. al. (PNAS 79:3523-3526 (1982)) and Mansfield (Magn. Reson. Med., 1(3):370-386 (1984)). In general, any method that determines the frequency distribution (chemical shift spectrum) at each spatial point may use the two specific embodiments above.

The methods provided herein detect and/or measure metabolite species with a high degree of specificity that allows one to obtain information concerning the presence of a disease state, progression of a disease state, the effect of treatment on the disease state, the selection of treatment for the disease state, and a prognosis of the disease, such as cancer.

### BRIEF DESCRIPTION OF THE SEVERAL VIEWS OF THE DRAWING

FIG. 1 shows the metabolic and catabolic pathways for the formation or precursors of various fatty acids (and/or esters of these fatty acids).

FIG. 2 shows the metabolic and catabolic pathways for the formation or precursors of various phospholipids.

FIG. 3A is a graph showing a typical 1D (one dimensional) proton spectrum at 600 MHz of a chloroform/methanol liver extract. FIG. 3B shows the chemical structure of one species of unsaturated fatty acid.

FIG. 4 is a graph showing changes in the glycerol backbone and “choline type compounds” concentration of rats fed a choline diet and those fed a choline-deficient diet over time.

FIG. 5 is a graph showing a continuum of the relative amounts of each of oleic acid, linoleic acid, linolenic acid and arachidonic acid dependent on the R value (R value is the ratio of the areas under the bis-allylic protons (the protons that resonate at 2.8 ppm) to those of the vinyl protons (the protons that resonate at 5.3 ppm)).

FIG. 6 shows a ¹H-¹³C HSQC pulse sequence employed in an embodiment of the present method wherein the upper pulses in the figure are pulses applied to the proton and the lower pulses in the figure are pulses applied to the X-nucleus (e.g., carbon). In this pulse sequence a hermite J pulse (90J) is used on the proton and a sech/tan h (S/T) pulse is used on the X-nucleus (in this example, carbon). The various delays (d1 and d2) can be modified dependent on the coupling constants between the spin linked sensitive and insensitive nuclei. After the final two pulses, one obtains reverse polarization transfer, gradient coherence selection by the gradients g1 and g2 and ultimately, the collection of the desired signal.

FIG. 7 is a graph showing the evolution of magnetization for various nuclei during J-pulse (the initial hermite proton pulse shown in FIG. 6) calculated on resonance using a 6×6 rotation matrix.

FIG. 8A is a graph showing a simulation of the polarization transfer that occurs in the pulse sequence shown in FIG. 6 to the end of the evolution period prior to any further evolution delay period. FIG. 8B is a graph showing a 6×6 rotation matrix simulation of the expected signal amplitude of the desired polarization transfer state, 2IzSx state of the hermite J pulse as a function of peak amplitude, RFmax.

FIG. 9 shows a 2D (two dimensional) ¹H-¹³C HSQC spectrum (1 ppm×5 ppm) of arachidonic acid obtained using the modified pulse sequence of FIG. 6.

FIGS. 10A-D show 2D (two dimensional) ¹H-¹³C HSQC spectras (1 ppm×5 ppm) of oleic acid, linoleic acid, linolenic acid and arachidonic acid.

FIG. 11A shows a 2D (two dimensional) ¹H-¹³C HSQC spectra of a mixture of 4 mg/ml oleic acid, 15 mg/ml linoleic acid, and 9 mg/ml linolenic acid. FIG. 11B shows a 2D (two dimensional) ¹H-¹³C HSQC spectra of a mixture of 11.2 mg/ml oleic acid, 30 mg/ml linoleic acid, and 12 mg/ml linolenic acid.

FIG. 12 shows an automated method algorithm used in an embodiment of the present method.

FIGS. 13A-D show the relative amounts over time of oleic acid, linoleic acid, linolenic acid and arachidonic acid in rats fed a choline-deficient diet and rats fed a diet that has adequate choline.

FIGS. 14A-D show general trends in the relative amounts over time of oleic acid, linoleic acid, linolenic acid and arachidonic acid in rats fed a choline-deficient diet and rats fed a diet that has adequate choline.

FIG. 15 shows ratio data (red contours) as calculated from spectra collected using a chemical shift imaging method overlaid on a T2 weighted image.

### DETAILED DESCRIPTION OF THE INVENTION

The method described herein is broadly directed to the detection of one or more metabolites that will allow for a determination of differences between a normal individual and an individual that has an elevated or lowered level of the metabolite or metabolites. In one embodiment, detection of a metabolite or metabolites in a sample obtained from an individual enables a determination of an adverse condition or disease state, such as a diagnosis of a type of cancer in the individual.

In one embodiment, the method utilizes magnetic resonance methodology for detecting metabolites that will allow one to determine if there are alterations between control and afflicted examination subjects that have distinguishable patterns of NMR signal and thus altered state of physiology. The magnetic resonance methodology can use either Nuclear Magnetic Resonance (NMR) spectroscopy, Magnetic Resonance Imaging (MRI), or both. Thus, in a variation of these embodiments, NMR spectroscopy and/or MRI can be used to detect alterations in metabolite signal patterns and their area and/or amplitudes, which in turn can be used to determine if examination subjects have biological condition/s characterized by the alterations in such signal patterns, area and amplitudes. It should be recognized by those skilled in the art that the above methodology is a general method wherein any magnetic resonance methodology can be used in the detection of the alteration metabolites. In order to more fully explain the method, it will be described in some detail with reference to ¹H-¹³C HSQC methodologies. It should be recognized that the method is a general method that allows the use of any element that has an NMR active isotope of that element.

Accordingly, when experiments, pulse sequences, and in general, the methodologies are described with reference to polarization transfer between carbon and protons, it should be recognized that any other nucleus that has an NMR active isotope of that element can be used in the method (as long as those elements are present in the metabolite to be observed).

An embodiment of the present method shows that with basic 1D (one dimensional) NMR spectra, the proliferation of compounds containing a glycerol backbone can be quantitated with high precision in a choline-deficient rat hepatocarcinogenesis model. Moreover, an embodiment of the present method allows determination of the concentration of compounds containing one or more double bonds, which is shown to be significantly elevated in the treated group. In addition, by taking the ratio of areas under the bis-allyl and vinyl peaks, it is possible to show a shift towards less double bonds with age and tumorigenesis. Although the methodology has been practiced and shown by applicants in one dimensional proton spectroscopy, the method was found to be limiting because when one compound dominates the signal it is very difficult to separate the minor signal components.

An embodiment of the present method investigates and confirms the possibility of disseminating species from the peaks at 5.3 ppm. Species may also be disseminated from any cluster of peaks in an NMR spectrum. Thus, the methods described herein are not limited to peaks at 5.3 ppm, but can be applied to peaks at any frequency where such signals occur because of slight differences in chemical structure of the metabolite. When the methods of selective spectroscopy are combined with in-line digital processing of the free induction decay, increased resolution is seen in a narrow selective bandwidth of spins. In this case, the vinyl methyl groups can be treated as a simple two coupled IS spin system, which lead to an embodiment of the present method using modified HSQC (heteronuclear single quantum coherence) pulse sequences.

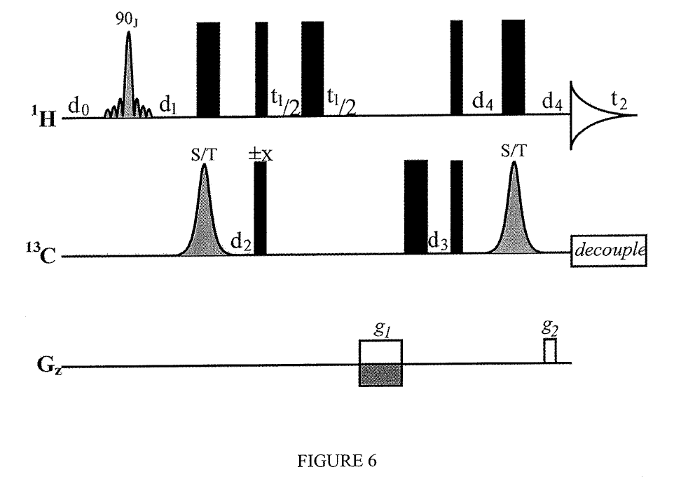

Traditionally, in NMR pulse sequence development; the trend has been toward finding the most efficient pulses of short duration to produce a particular spin state in high yield. These strategies have included numerical analysis of the Bloch equations, treatment of an NMR spin system in the quantum mechanical paradigm, vector analysis, and optimal control theory applied to the Bloch equations. Often these pulses are designed for broadband application and excite off-resonance spins that are irrelevant to embodiments of the present method that look at narrow spectral widths. The general concept of this embodiment is similar to spin pinging where a selective set of resonances are excited rather than a single line of a multiplet structure. With the use of a J-pulse (see FIG. 6), a desired spin state can be achieved by the end of a pulse and the pulse may be applied through the complete evolution delay period in the conventional NMR experiment with minimal loss of signal amplitude by the end of the sequence provided that due compensation is made for complete evolution by extension of the delay period d1 as shown in the diagram of FIG. 6.

Thus in HSQC, to create a 2I_{z}Sₓ spin state, a pulse may be applied for the entire ¼J delay period and still produce in high yield that state. Also in HSQC, the INEPT (Insensitive Nucleus Enhanced by Polarization Transfer) type transfer utilizes the inversion of the attached spins to refocus the scalar coupling evolution. This is advantageous for two reasons. First, when inverting spins, it is possible to achieve very high selectivity across an effective spectral bandwidth without affecting resonances outside a chosen spectral width. Second, this rotation can be achieved adiabatically in the true sense and so contributes to the robustness of the method.

In NMR, after a pulse or series of pulses, a number of quantum states can be rationalized. Of interest in the methods provided herein are the single quantum states for two interacting spin states—2IzSx, 2IzSy, and 2IzSz (i.e., the interaction between a sensitive nucleus S and an insensitive nucleus I). By using one of or both of phase cycling and one or more gradient pulses, coherence selection conditions can be obtained that allow for the observation of a given particular resonance. Generally, phase cycle methods take longer but give very clean signal selection whereas gradient pulses tend to shorten the acquisition time relative to non-gradient phase cycling for an NMR experiment (or an imaging experiment) but have the minor drawback of a concomitant loss of signal which nevertheless may be re-gathered with more pulses.

The methods provided herein are also described with reference to fatty acid/lipid/phospholipid metabolites and the measurement of concentration changes of those fatty acid/lipid/phospholipid metabolites. It should be recognized, however, that other possible metabolites can be used in the methodology of the present methods, including but not limited to proteins, nucleic acids, vitamins, peptides, sugars, amino acids, and steroids.

Accordingly, in a variation of an embodiment of the methods described herein, the levels of oleic acid, linoleic acid, linolenic acid and arachidonic acid (and/or the esters of each of these fatty acid derivatives) were measured using the present method. Thus, in an embodiment, the method employs spectral data acquisition and analysis for the complete and accurate analysis of at least four predominant species containing double bonds associated with a fatty acid moiety. Changes in the concentrations of these fatty acid/lipid/phospholipid metabolites are indicative of a biological condition in an individual, and in particular, the presence of the early stages of cancer. Accordingly, the method provided herein is advantageous in that it allows one to determine early stages of biological conditions such as cancer, thereby facilitating early treatment and enhancing recovery or remission.

### Descriptions of Figures

The methods provided herein will also be described with reference to the figures. This description is solely for the sake of understanding the methods and should not be construed to limit the invention to the particular embodiments described.

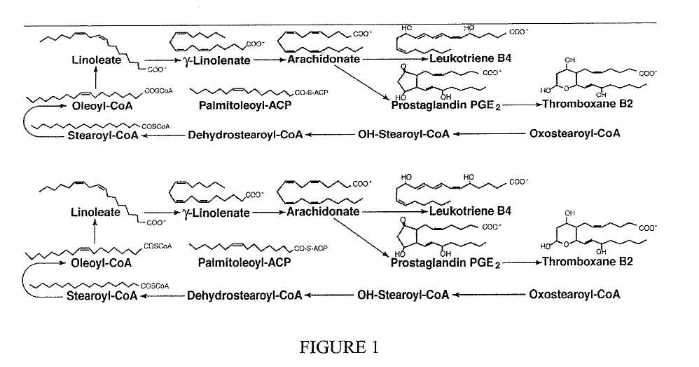

FIG. 1 shows the metabolic and catabolic pathways for the formation or the precursors of the various fatty acids (and/or esters of these fatty acids). This figure shows the relative numbers of double bonds in each of the fatty acids and the precursors and products of each of the respective fatty acids. The relative amounts of certain of the fatty acids show that a biological condition exists in an individual that has either an elevated or lowered concentration of one or more of these fatty acids (to be explained in more detail below).

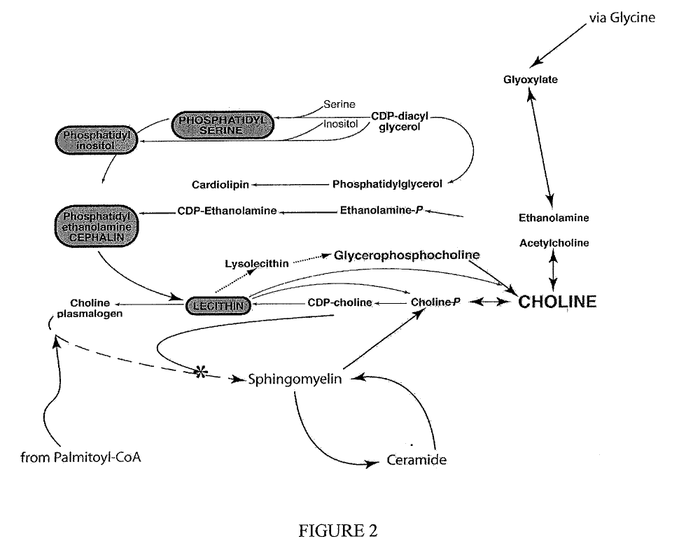

FIG. 2 shows the metabolic and catabolic pathways for the formation or the precursors of the various phospholipids. The relative amounts of certain of the various phospholipids show that a biological condition exists in an individual that has either an elevated or lowered concentration of one or more of these phospholipids.

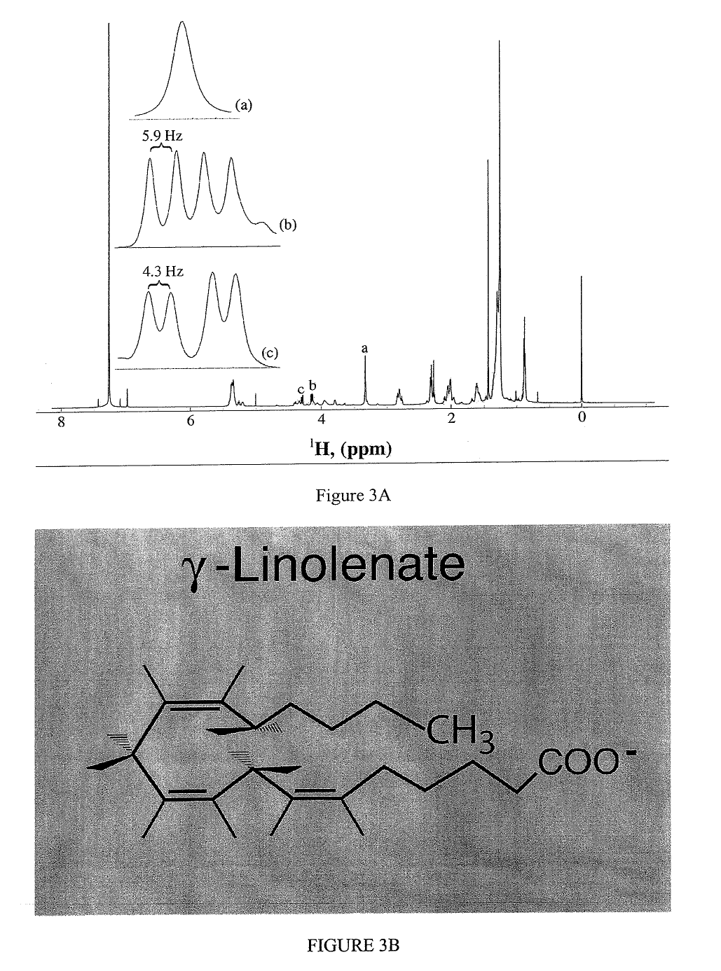

FIG. 3A shows a 1D proton spectrum at 600 MHz of a chloroform/methanol extract of liver tissue which contains compounds such as, for example, γ-linolenate whose chemical structure is shown in FIG. 3B. Of particular interest in this 1D proton spectrum are the protons that are the bis-allylic protons (the protons that resonate at 2.8 ppm and the vinyl protons (the protons that resonate at 5.3 ppm). The bis-allylic protons (or protons comprising methylene groups) are those that are present at carbon positions 8, and 11. The vinyl protons (or protons comprising methine groups) are present at carbon positions 6, 7, 9, 10, 12, and 13 of the fatty acid class of compounds. The relative ratios of these two sets of protons give some indication of the number of double bonds that are present in the chemical species of a class of compound such the fatty acids and its many derivatives that could be present. As the number of double bonds decrease, the vinyl protons also decrease (and the area under the peaks at about 5.3 ppm also decreases). Likewise the area under the 2.8 ppm peak also decreases.

The relative amounts of various species of fatty acids that are present can be ascertained by magnetic resonance techniques (these relative amounts are indicative of a biological condition present in the examination subject). The relative amounts of the fatty acids species that are present can be ascertained by comparing the ratio of the relative areas under the peaks at 2.8 and 5.3 ppm.

The relative amounts of various species of fatty acids can be surmised by the following relationship: Number of double bonds=−1/(R−1) where R is the ratio of the area under the bis-allylic and vinyl protons that can be deduced by relating the relative concentrations of protons in each group contributing to the NMR resonance. Table 1 below gives an overview of the fatty acid species likely to be present based on the R value.

TABLE 1

| Fatty acid | Ratio (R) | Double bonds |
|---|---|---|
| Oleic | 0 | 1 |
| Linoleic | 0.5 | 2 |
| Linolenic | 0.6 | 3 |
| Arachidonic | 0.75 | 4 |

This table demonstrates that when the R value is 0 or a value very close to 0, most of the fatty acid chemical species is likely to contain only one double bond such as oleic acid. Likewise, if the R value is a value that is about 0.75, much of the fatty acid chemical species present is likely to contain 4 double bonds such as arachidonic acid.

The differences in these R values and the relative fatty acid species dependent upon these R values were advantageously used in one of the embodiments of the present method. Two rat groups were used. The first group consisted of rats that were sustained on a diet sufficient of choline (CSAA) and a second group of rats were sustained on a choline-deficient diet (CDAA). As the rats in the CDAA group aged, they developed nodules and/or tumors in their liver, which eventually led to liver cancer. During the time of feeding of the rats, a number of changes occurred, and these can be studied by the NMR spectroscopy and MRI spectroscopic imaging techniques described within.

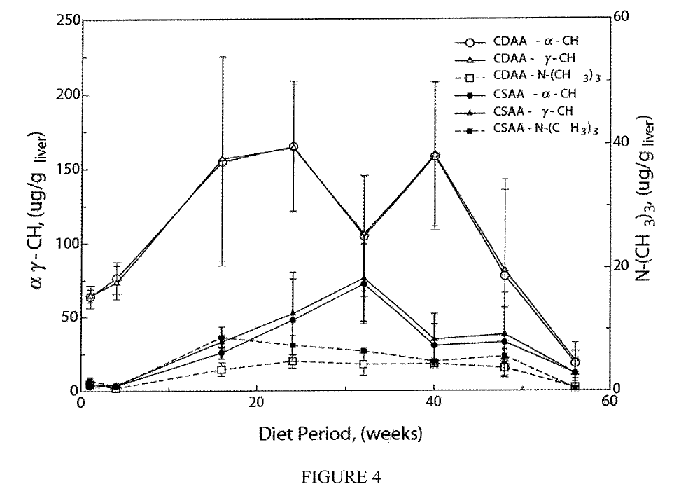

Some of the changes in the rats are apparent by an observation of FIG. 4. FIG. 4 shows the changes in the glycerol backbone concentrations of rats fed a choline diet and those that were fed a choline-deficient diet over time. FIGS. 1 and 2 show the metabolic and catabolic pathways of fatty acids and phospholipids (with a glycerol backbone). FIGS. 1 and 2 are interrelated as one of the precursors to phospholipids is palmitoyl CoA, and palimitoic acid is one of the fatty acids shown in the pathway of the fatty acids. Accordingly, the availability of palmitoic acid is likely to influence the metabolic and catabolic pathways of the glycerol backbone. Thus, there does appear to be a relationship between the fatty acid biosynthetic pathway and the corresponding biosynthetic pathways of phospholipids.

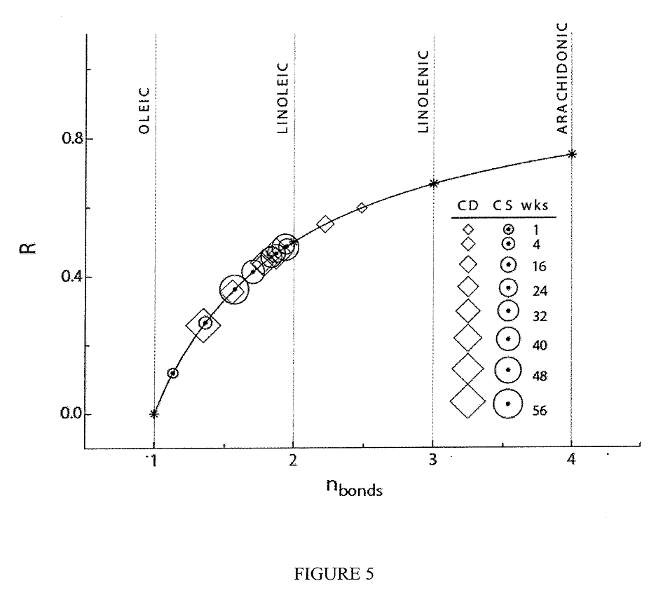

FIG. 5 shows a continuum of the relative amounts of each of oleic acid, linoleic acid, linolenic acid and arachidonic acid dependent on the R value. The R value is the ratio of the areas under the bis-allylic protons (the protons that resonate at 2.8 ppm) to those of the vinyl protons (the protons that resonate at 5.3 ppm). The continuum is shown with regard to the general trends that can be observed in the groups of rats that are fed both a choline diet and those that are fed a choline-deficient diet.

FIG. 6 shows the ¹H-¹³C HSQC pulse sequence employed in the present method wherein the upper pulses in the figure are pulses applied to the proton and the lower pulses in the figure are pulses applied to the X-nucleus (e.g., carbon). In this pulse sequence, a hermite J pulse is used on the proton and a sech/tan h pulse is used on the X-nucleus (in this example, carbon). The various delays (d1 and d2) can be modified dependent on the coupling constants between the spin linked sensitive and insensitive nuclei. After the final two pulses, one obtains reverse polarization transfer, gradient selection and, ultimately, collection of the desired signal.

Because biological samples contain a plurality of different resonances, and the proton spectrum does not have a very large chemical shift range, overlapping resonances may prove to be problematic in ascertaining a particular resonance without the interference from these overlapping resonances. One way of adjusting for these overlapping resonances is by the use of selective magnetization transfer from a spin coupled X-nucleus by the creation of magnetization transfer conditions that will target a given particular proton resonance (while consequently not targeting the overlapping proton resonances that have similar chemical shifts). In other words, by employing an HSQC type experiment (or alternatively another magnetization transfer experiment) one can “filter” unwanted resonances and observe only those proton resonances that are of interest.

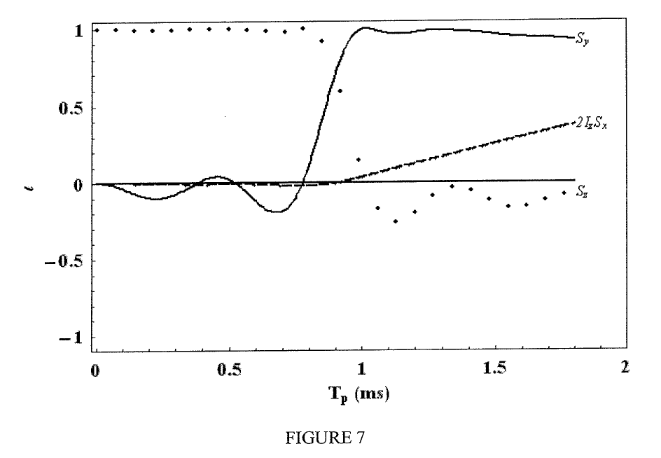

FIG. 7 shows the evolution of magnetization for the various nuclei during the J-pulse (the initial hermite proton pulse shown in FIG. 6) calculated on resonance using a 6×6 rotation matrix.

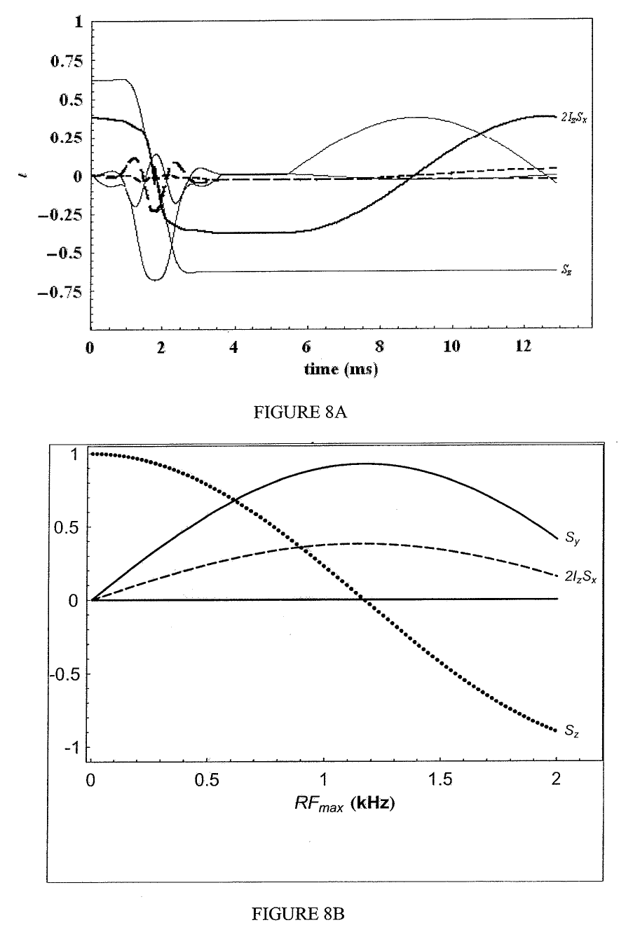

FIG. 8A shows a simulation of the polarization transfer that occurs in the pulse sequence shown in FIG. 6 to the end of the evolution period. FIG. 8B shows a simulation of the peak amplitude, RFmax, and the generation of the desired 2IzSx state by the J-pulse.

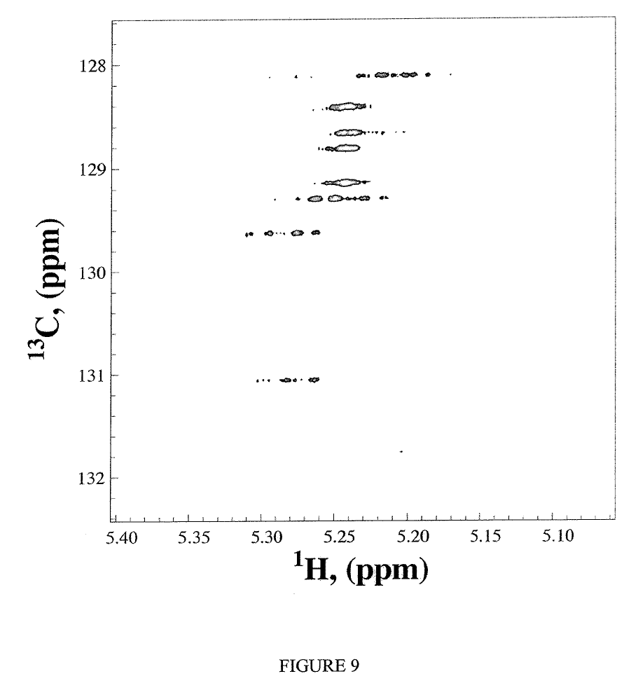

FIG. 9 shows that selective magnetization conditions can be achieved that allow indirect detection of the proton resonances (correlated with their spin coupled carbon atoms) in a fatty acid. FIG. 9 shows a 2D ¹H-¹³C HSQC spectrum (1 ppm×5 ppm) of arachidonic acid.

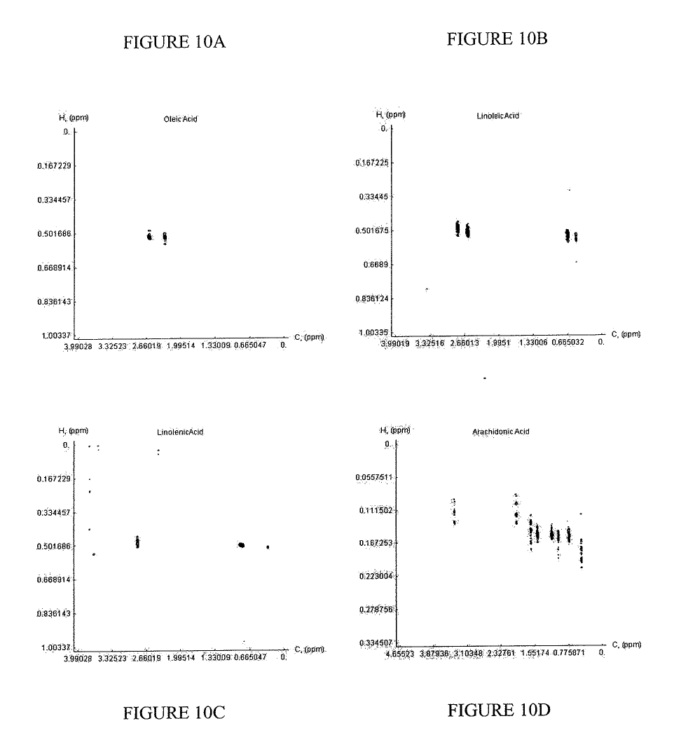

Likewise, FIGS. 10A-D show that a 2D ¹H-¹³C HSQC spectra (1 ppm×5 ppm) can be obtained for all of oleic acid, linoleic acid, linolenic acid and arachidonic acid

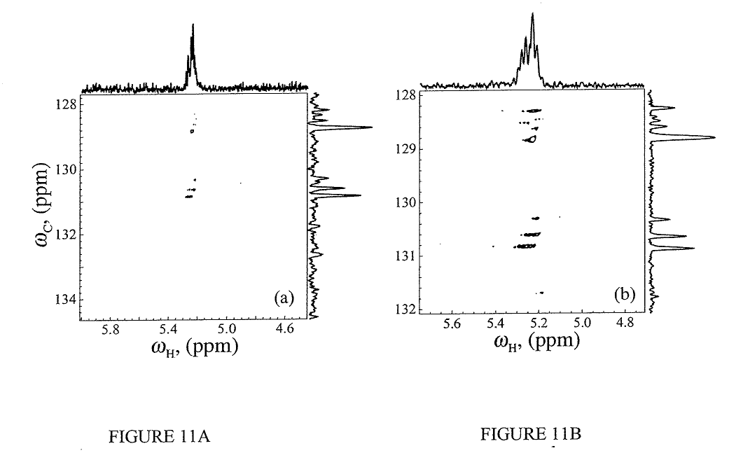

Subsequent to showing that an individual spectrum of a given fatty acid can be obtained, it was desired to show that a mixture of fatty acids can use the procedures of the present method. Accordingly, FIGS. 11A and B show 2D ¹H-¹³C HSQC spectra of two mixtures of oleic acid, linoleic acid, and linolenic acid with FIG. 11A represented by a mixture that is 4 mg/ml oleic acid, 15 mg/ml linoleic acid, and 9 mg/ml linolenic acid and FIG. 11B being obtained from a mixture that has 11.2 mg/ml oleic acid, 30 mg/ml linoleic acid, and 12 mg/ml linolenic acid.

Table 2 shows the results in tabular form of the mixtures of the oleic acid, linoleic acid, and linolenic acid as calculated by the methodology of the present method (using NMR spectroscopy) versus the actual measured concentrations of each of the respective fatty acid samples.

TABLE 2

| | | Actual | Calculated | Deviation | |
|---|---|---|---|---|---|
| | | Conc., | Conc., | from | |
| Sample | Compound | [mg/ml] | [mg/ml] | Actual | Std. Conc. |
| Test Mix. 1 | Oleic | 4.0 | 3.5 | −12.5 | 5.0 |
| | Linoleic | 15.0 | 17.6 | 17.3 | 5.0 |
| | Linolenic | 9.0 | 11.8 | 31.0 | 3.0 |
| Test Mix. 2 | Oleic | 11.2 | 8.2 | −27.0 | 5.0 |
| | Linoleic | 30.0 | 29.3 | 2.3 | 5.0 |
| | Linolenic | 12.0 | 12.1 | 0.8 | 3.0 |

A general automated method would be beneficial in detecting the relative amounts of the given fatty acids. The method would involve identifying the peak as one of the target peaks, removing any noise from the spectrum, calculating the area and concentration of the peak and checking that the peak quantitated is actually the desired peak and that it correlates with one of the standard peaks (in a pure sample containing only that fatty acid). One of the potential limitations of the present method is that chemical shifts do change ever so slightly dependent upon a number of factors, such as the relative concentrations of the metabolites being observed, other metabolites which may be in the mixture, various solvent effects, etc. These effects can be minimized by consistent sample preparation or alternatively can be adjusted for by having a large number of standards on which one can base chemical shift data.

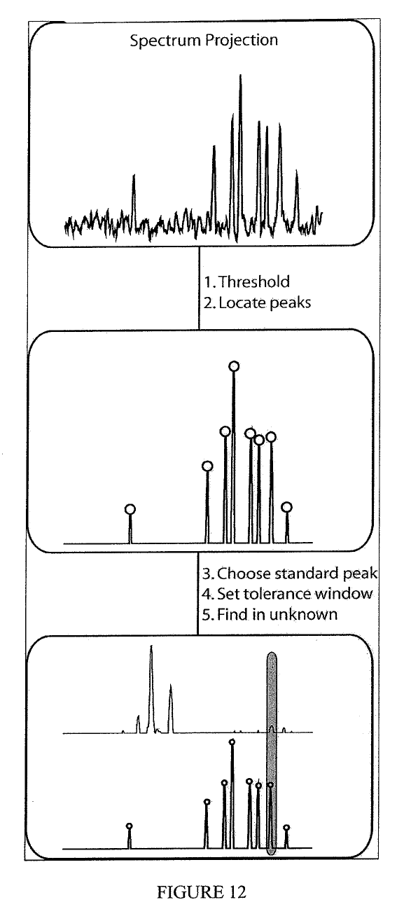

FIG. 12 shows an automated method for determining the relative amounts of metabolites at 4 weeks, 24 weeks and 56 weeks after the feeding of the rats, respectively. After collection of the spectra, concentrations are determined in an automated fashion using the algorithm schematized above. The maximum projection intensity spectrum of the indirect dimension is first constructed (1). A threshold level is determined and peaks appearing in the spectrum are determined (2) and their coordinates stored. This is kept constant for each sample by employing constant instrument parameters between samples. From a database of standard peaks (3) a target resonance is selected. The tolerance window is chosen and set (marked by the grey region) (4) and located by a “search” routine in the unknown (5).

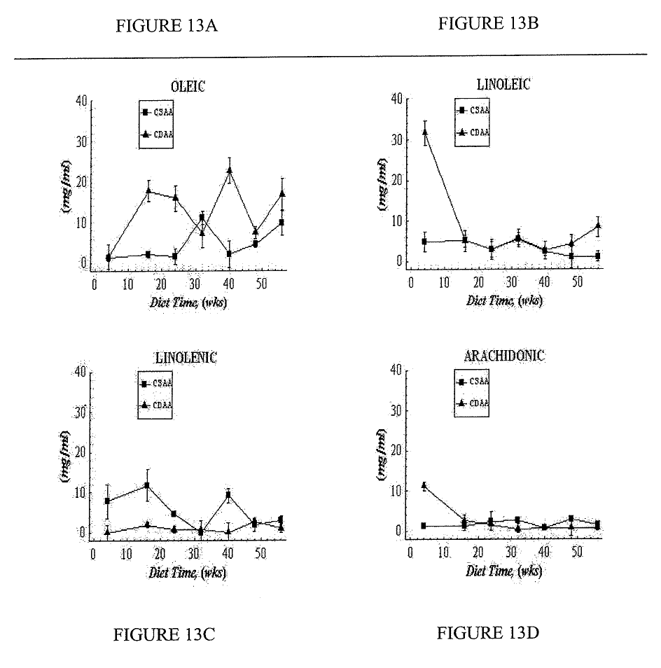

FIG. 13 shows the relative amounts of oleic acid, linoleic acid, linolenic acid and arachidonic acid in rats fed a choline-deficient diet and rats fed a diet that has adequate choline over time. It should be noted that the rats that are fed the choline-deficient diet have elevated levels of oleic and linoleic acid after a year relative to those rats that are fed a diet containing choline. Accordingly, it can be seen that there is a correlation between the levels of these fatty acids and the development of tumors in rats (i.e., tumors in rat livers were known to develop in rats fed a choline-deficient diet).

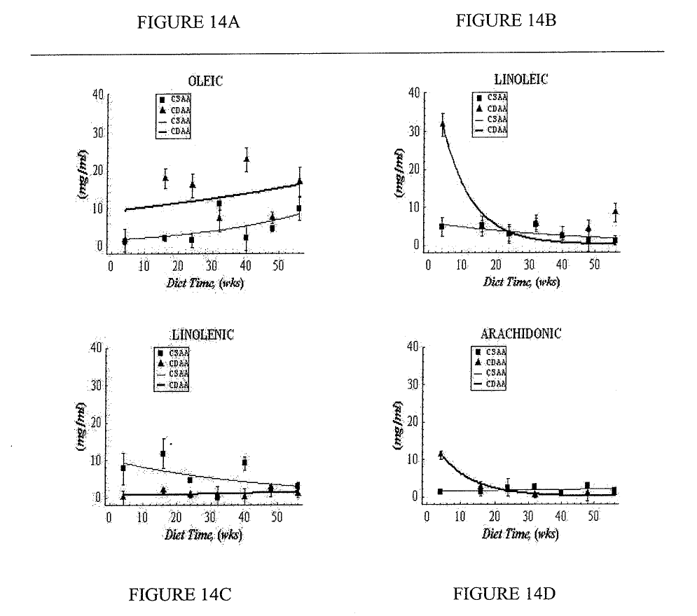

Lipid modeling data and the general trends of lipid metabolism is better seen when the data from FIGS. 13A-D are modified to generate FIGS. 14A-D. In other words, FIGS. 14A-D show general trends in the relative amounts of oleic acid, linoleic acid, linolenic acid and arachidonic acid in rats fed a choline-deficient diet and rats fed a diet that has adequate choline over time.

By using the data, for example, from FIG. 13 and employing a polarization transfer magnetic resonance experiment in rat liver samples (such as by employing the pulse sequence shown in FIG. 6), one can detect the early stages of tumor formation or hepatocarcinogenesis. In other words, by observing elevated levels of oleic and linoleic acid, one is likely to find rats that are in the early stages of tumor formation or hepatocarcinogenesis.

The above described methods will work on other animals, such as humans and other animals, which will lead to the early detection of tumors or carcinogenic conditions in those animals that are tested. Moreover, although the above conditions were described with reference to livers, it should be understood that the above method is a general method that can be advantageously used in any of a number of organs or places in which carcinogenesis is likely to occur including but not limited to ovarian, breast and brain cancers.

One minor drawback to the powerful methodology disclosed above is that samples must be gathered in order to subject them to polarization transfer experiments. Accordingly, if a tissue sample is to be collected from an individual this may require a biopsy in order to secure a sample. With this in mind, one advantageous embodiment of the present method is directed to non-invasive resonance imaging. This allows the detection of elevated levels of metabolites by observing a magnetic resonance image. This technique will preclude the use of collecting samples using an invasive procedure such as by biopsies.

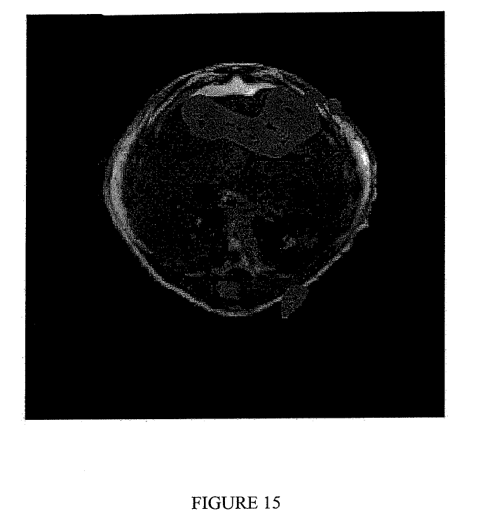

Of potential are methods such as CSI (chemical shift imaging) and quantum coherence selected methods. One embodiment of the present method is directed to imaging using a spin echo CSI method where the ratio R is calculated from each voxel of the CSI data set. FIG. 15 provides a transgenic mouse model of hepatocarcinogenesis, and shows that the distribution of compounds containing fatty acids with a number of double bonds can be detected and mapped over the liver. The contours in FIG. 15 are when 0.2<R<0.6 corresponding to compounds containing 1½ to 2½ double bonds and are overlaid on the morphological T2 weighted image of the liver. This result is consistent with previous finding of applicants that, with age and hepatocarcinogenesis, the number of compounds with one double bond dominates the lipid profile of the liver (described above). Magnetic field homogeneity maps should be established to generate confidence in the chemical shifts mapped. The use of phantoms, by incorporating gradient shimming and a user controllable CSI weighting scheme, shows excellent linewidths and chemical shift stability of a chosen resonant signal. The use of selective RF pulses on the bis-ally and vinyl resonances provides convenient in vivo methods for the detection of fatty acid species distribution in the liver during hepatocarcinogenesis.

### Identification and Quantification of Peaks in Samples

While there are many methods for determining the peaks in unknown samples, the most popular being principle component analysis, such methods are based on the assumption that all peaks are unknowns. In the methods described herein however, comparison with standard compounds facilitates unequivocal assignment. It must also be noted, that in the analysis of numerous spectra (hundreds in this case), the instrument parameters should be kept constant for standards and samples, for the sake of reproducibility. In this way, software routines can be written to automate the analysis procedure. The algorithm used in an embodiment of the present method is presented in FIG. 12 for convenience.

Typical HSQC spectra of pure standards collected individually using the modified sequence is shown in FIG. 10 with a chemical shift scale to demonstrate the narrow bandwidths within which the fatty acid signals of interest resonate. The spectra for the three fatty acids, oleic, linoleic, and linolenic were collected with the same parameters and with a spectral width of 600 Hz and the spectra for arachidonic acid was collected with a slightly larger spectral width to avoid folding of signals in the ¹³C dimension and is thus shown separately. Sample spectra were collected using the same parameters as for arachidonic acid and as described above. The ability to reduce contribution of signals from other parts of the spectrum by using the selective hermite and sech/tan h pulses is clearly demonstrated in the spectra of FIG. 10. In the proton dimension, extensive homonuclear coupling can be seen. The collapse of these couplings was not sought with constant time methods or decoupling pulses during a mixing period, however, it is entirely possible to do so, and thus, this is a variation of an embodiment of the present method. This would benefit the sequence greatly by allowing an additional dimension for complete and unequivocal assignment of resonances to a particular fatty acid.

Second dimension maximum intensity projection spectra from a test mixture are presented in FIG. 10. Even though considerable overlap of peaks is evident, at least one resonance can be selected for the species oleic, linolenic and arachidonic acids to serve as a reference standard. The concentration of linoleic acid can be determined from the overlapping resonances of linoleic and linolenic fatty acids. Concentrations reported here are corrected for this overlap. With regard to sensitivity, it is apparent that because of the variable peak heights and the chosen reference, linolenic is the most insensitive.

External standards were used to identify peaks from pure standards, the goal was to account for those peaks in the unknown spectra given some tolerance for differences in chemical shifts as noted above. As a first step, the pure standards spectra were used to create a database of expected chemical shifts after applying a threshold to the data. One peak from each fatty acid species corresponding to the number of double bonds was chosen to serve as the standard for that species. These are highlighted for convenience in FIG. 13. The concentration of found peaks in samples was determined according to the following formula,

(Aᵤₙₖ/A_{std})×[X, mg/ml],

where Aᵤₙₖ is the area under the unknown sample peak, A_{std} is the area under the standard sample peak and X is the concentration of the known fatty acid standard.

Results for the two test mixtures are presented in Table 2. Calculated concentrations of fatty acids in the mixtures are in reasonable agreement with expected concentrations. Based on the deviations presented here, the accuracy of the method can be guaranteed to better than 70%.

In the INEPT step of the HSQC pulse sequence, the product operator state 2I_{z}Sₓ is created by the end of the first delay period preceding the application of any refocusing or inversion pulse and RF pulses are usually chosen so that they are of short duration. In this respect, the pulses used are said to operate at the “high power limit”, where coupling evolution during the pulse can be ignored and coupling subsequently evolves in a delay period set to match the evolution period via the scalar coupling constant. When a long low power pulse is used, such as the hermite pulse in FIG. 6, coupling evolution during the pulse cannot be ignored. These modifications however, lead to losses in signal amplitude by the end of the sequence, and in consequence, long, low power pulses are usually avoided in high resolution NMR. From the point of view of selective spectroscopy over very narrow bandwidths in crowded regions of a spectrum, the use of such pulses are inevitable, and, no matter what the pulse, the narrower the chosen excitation profile across an effective bandwidth, bw_{eff}, the longer the pulse duration Tₚ, because bw_{eff}α1/Tₚ. In addition, while adiabatic inversion pulses are now common place in spectroscopy applications, there is a tendency to use pulses optimized for shorter duration, such as WURST and tan h/tan pulses. Again, the selectivity of these pulses is determined by the duration Tₚ and the initial experiments that were done leading up to the present invention have found the sech/tan h pulse to yield very selective profiles and still retain most of its adiabatic character provided the pulse parameters of Tₚ, peak amplitude, RFₘₐₓ, and extent of the frequency sweep, bwdth are chosen to fall within the definition of linear adiabaticity.

A minor disadvantage of this selective HSQC pulse sequence is that homonuclear coupling cannot be ignored and complicated coupling patterns are observed, particularly for spin systems such as that found in arachidonic acid. There is, of course, additional information that can be derived from such coupling constants, which were not explored by Sandri et al. ((Sandri J, et al. Magn. Reson. Chem. 1997; 35:785-794.), but may be useful in future studies. These coupling constants provide valuable information on the length of the saturated chains terminated by the methyl and carboxyl groups and add an extra dimension for accurately determining species. Accordingly, in an embodiment of the present method, the coupling constants can be used to provide additional valuable information to ascertain that the correct peak is being observed. One drawback of these determinations is that these experiments may take on the order of hours to acquire.

Generally, to practice the present methods, samples should be prepared as consistently as possible. If consistent sample preparation is undertaken, one can utilize a similar set of shims and linewidths of each resonance to identify resonances and quantification rarely exceeds a prescribed limit. In one embodiment, the present method uses the criteria of less than 1% deviation, at half height, of the proton signal from chloroform, which has been described in detail previously. For this reason, resonances may be assigned automatically with little error and peak areas can be measured consistently. If these procedures are followed, accurate quantification can be made.

In an alternative embodiment, internal references can be used to deduce sample concentrations. Alternatively, a single point external reference standard from pure compounds can be used. If a single point external reference standard is used, the method may be limited to an extent by large deviations from calculated and expected concentrations. However, the beneficial effects of short collection times may be a reason for choosing a single reference standard for quantification. For translation to an in vivo setting the internal standard may be too time consuming to acquire spectra from various concentration standards to create a calibration curve. In any event, it is expected that the accuracy of this method can be guaranteed to greater than 70% of the expected concentration in a biological sample.

The separation of different fatty acid groups in phospholipids within a biological sample is of diagnostic potential, as it has been shown that alterations in fatty acyl group composition, particularly the unsaturated fatty acids, are associated with various cancers. For instance, a loss of stearoyl-CoA desaturase (Scd) expression is a frequent event in prostrate carcinoma. Scd catalyzes the rate limiting step in the synthesis of monounsaturated fatty acids, as oleate (C10:1), from the desaturation of saturated fatty acids. An alteration in saturated to monounsaturated fatty acid composition has been implicated in a number of disease states including cardiovascular disease, obesity, diabetes, neurological disease, and cancer (Ntambi J M, et al., Pro. Lipid. Res. 2004; 43:91-104.). Also ω6-desaturase has been shown to be deficient in malignant melanoma cells, prostate tumors and breast cancer. ω6-desaturase is involved in the initial step in the conversion of omega-3 and omega-6 linoleic acids. The fatty acid speciation method disclosed in this invention allows the evaluation of key metabolite processes such as fatty acid desaturation that may be related to a pathological process such as cancer. Thus, in an embodiment, the present method provides for the specific determination of enzymatic or genetic events associated with progressive stages of a disease, which may be potentially used as an early diagnostic method for diseases that undergo alterations in fatty acid metabolism, such as cancer or diabetes.

The present method describes the use of a modified HSQC pulse sequence for determining the species and concentration of fatty acid species that are generally thought to be difficult to resolve by NMR spectroscopy. By using narrowband pulses specifically tailored to operate on discrete ranges of the chemical shift spectrum, signals from other parts of the spectrum can be suppressed efficiently. This allows for collection of selective spectra with reduced points in both the direct and indirect dimensions thus affording better resolution in the final spectrum. Accordingly, overlapping peaks can be resolved, identified and quantified.

Likewise, the imaging aspects of the present method should allow overlapping peaks to be resolved, identified and quantified on an image. As an example that the CSI methodology works, FIG. 15 shows the gradient echo T1 image of cirrhotic liver in a TGF-alpha/cmyc transgenic mouse (bottom) showing abnormal structure and signal brightness due possibly to tumor formation. On the top image, a contour plot of the ratio, 0.2<R<0.6, is calculated from individual voxels of a spin echo chemical shift imaging experiment are shown overlaid on a T2 weighted image of the same slice as in the gradient echo. Accordingly, the imaging aspects of the present method should allow a non-invasive determination of the presence of hepatocarcinogenesis.

The methodologies discussed above have been shown with a particular example using differences in concentrations of fatty acids or glycerol backbones to show the presence of hepatocarcinogenic conditions. It should, however, be recognized that these disclosed methodologies are general methodologies which should be adaptable to the detection of any of a number of metabolites. The detection of a change of any of these metabolites will allow one to deduce the changes in biological conditions of an individual that possesses that certain biological condition.

Thus, in an embodiment, the present method is a method of detecting a change in concentration of one or more metabolite markers from a first sample to a second sample, wherein the method comprises: identifying one or more metabolite markers to study; using nuclear magnetic resonance spectroscopy or magnetic resonance imaging to indirectly detect the metabolite markers in the first sample and the second sample; comparing the concentration of the metabolite markers from the first sample and the second sample; and detecting the change in concentration from the first sample to the second sample.

In a variation of this embodiment, the present method uses nuclear magnetic resonance imaging. The magnetic resonance imaging in a variation uses a methodology and the necessary corresponding pulse sequence wherein one or more metabolite markers are detected by chemical shift imaging. In an alternate variation of the embodiment, the present invention is directed to NMR spectroscopic methods wherein the one or more metabolite markers are indirectly detected using one or more of HSQC, HMQC, HMQC-NOESY, HMQC-TOCSY, HMBC, or INEPT.

In an embodiment, the metabolite marker is a fatty acid, a protein, a nucleic acid, a vitamin, a peptide, a sugar, an amino acid, a phospholipid, a steroid or combinations thereof.

In an embodiment, the sensitive nucleus is a proton and the insensitive nucleus is carbon.

In one variation, where fatty acids are used as the metabolite markers, the metabolite markers are oleic acid, linoleic acid, linolenic acid, or arachidonic acid.

The samples to be tested are derived from an animal such as a mammal and include, but are not limited to, humans or other primates; rodents such as rats or mice; domesticated animals such as dogs, cats, ferrets, and guinea pigs; livestock such as cows, pigs, sheep, or horses, or other animals. Thus, the methodologies of the present methods should not only be efficacious for medical purposes by medical physicians but are also efficacious for use by veterinarians.

In an alternate embodiment, the present invention is directed to a method of detecting a biological condition in an individual by identifying one or more metabolite markers that are known to change in concentration when the biological condition is present; using nuclear magnetic resonance spectroscopy or magnetic resonance imaging to indirectly detect the metabolite markers; comparing the concentration of the metabolite markers from when the biological condition is present to when the biological condition is not present; thereby detecting the biological condition.

In a variation of this embodiment, the biological condition is cardiovascular disease, obesity, diabetes, neurological disease or cancer.

In a variation of this embodiment, the method uses nuclear magnetic resonance imaging. The magnetic resonance imaging uses a methodology and the necessary corresponding pulse sequence wherein one or more metabolite markers are detected by chemical shift imaging. In an alternate variation, the NMR spectroscopic methods are used wherein the metabolite markers are indirectly detected using one or more of HSQC, HMQC, HMQC-NOESY, HMQC-TOCSY, HMBC, or INEPT.

In an embodiment, the metabolite marker is a fatty acid, a protein, a nucleic acid, a vitamin, a peptide, a sugar, an amino acid, a phospholipid, a steroid or combinations thereof.

In an embodiment, the sensitive nucleus is a proton and the insensitive nucleus is carbon.

In one variation, where fatty acids are used as the metabolite markers, the metabolite markers are oleic acid, linoleic acid, linolenic acid, or arachidonic acid.

In one variation, where fatty acids are used as the metabolite markers, the fatty acids are used to detect a cancer such as, but not limited to, prostate, ovarian, breast, brain, and liver cancer. In an embodiment, the cancer detected is liver cancer.

The samples to be tested are derived from an animal such as a mammal and include, but are not limited to, humans or other primates; rodents such as rats or mice; domesticated animals such as dogs, cats, ferrets, and guinea pigs; livestock such as cows, pigs, sheep, or horses, or other animals. Thus, the methodologies of the present methods should not only be efficacious for medical purposes by medical physicians but are also efficacious for use by veterinarians.

Thus, in an embodiment, the present method identifies fatty acid species based on the number of double bonds contained in a lipid molecule. Common to all polyunsaturated fatty acids are two signature resonances occurring at approximately 5.3 and 2.8 ppm in the proton chemical shift spectrum of nuclear magnetic resonance (NMR), which can be detected by the methodologies disclosed in the present method. In a variation of this embodiment, the present method utilizes a modified conventional HSQC pulse sequence with a J-pulse on the spin system of the vinyl group (generalized as an IS spin system), at the beginning of the initial polarization transfer period and selectively inverting the ¹³C (I) spins with a narrowband sech/tan h inversion pulse. The method allows for the collection of data in both nucleus dimensions and can be restricted to a narrow slice of the chemical shift range. Accordingly, when these narrow chemical shift ranges are used, the resolution is subsequently determined by digitizer efficiency and spectra can be collected within a 1×6 ppm window of the respective proton and carbon chemical shift ranges. With this modification it is possible to distinguish at least one resonance each from the multiple shifts expected from the indirectly detected nuclei of the fatty acid species, oleic, linoleic, linolenic and arachidonic acids, which contain 1, 2, 3 and 4 double bonds, respectively. This and similar methods of applied selectivity are disclosed that show universal applications in characterizing speciation in biological samples where mixtures are often encountered and chemical shifts of the same structural group of similar molecules give rise to complicated overlapping resonances but are important for diagnosis of disease processes such as cancer.

The methods described herein will be further understood with reference to the following non-limiting example.

### EXAMPLE 1

### HSQC Experiment

A gradient HSQC experiment (Kay L. E., et al., J. Am. Chem. Soc. 1992; 114: 10663-10665) without sensitivity enhancement was modified to include a hermite J-pulse (Bendall M. R., et al. J. Magn. Reson. 1999; 141:261-70.) of duration Tₚ=1.8 ms and a narrowband sech/tan h (Silver M. S., et al., Phys. Rev. A. 1985; 31:2753-2755.) inversion pulse of duration 3.6 ms as shown in FIG. 6. The peak amplitude, RFₘₐₓ of the hermite pulse was calculated by simulation using the 6×6 rotation matrix as illustrated in FIG. 7, assuming a one-bond scalar coupling constant between the vinyl proton and the attached heteronucleus of J=140 Hz, and was chosen to be 1.2 kHz corresponding to the maximum of the 2I_{z}Sₓ curve of the plot in FIG. 8. Parameters for the sech/tan h inversion pulse were determined using universal equations previously published (Tesiram Y. A., et al., J. Magn. Reson. 2002; 156: 26-40.). Briefly, the duration of this pulse was chosen to occupy the entire ½J evolution period in the initial INEPT period of HSQC, assuming again that J=140 Hz. With this duration, the minimum bandwidth bwdth, of the pulse was determined to satisfy the condition for linear adiabaticity where, Tₚbwdth>10. The maximum inversion efficiency sought was for greater than 95% inversion, i.e. t₀>0.9. Using Equation 8 of Ref. 6, RFₘₐₓ=1.24 kHz for the sech/tan h pulse and to ensure >95% inversion an additional 5% of RFₘₐₓ was added to that and calculated. The same inversion pulse was used in the reverse INEPT step of the HSQC pulse sequence. All other pulses used were hard pulses. Proton 90° pulse durations were determined to be 4.2 μs and ¹³C pulses were determined to be 13.5 μs. The duration's d₁ and d₂ were inserted into the pulse sequence and accommodated gradients for coherence preparation and de-phasing of unwanted coherences. Their lengths were determined by experiment using a standard sample of 10 mg/ml arachidonic acid in deuterochloroform. Maximum signal was observed when d₁=0 (exclusive of any gradients pulses applied) and when d₂=2.7 ms.

### Standards and Test Mixtures

Standard samples of pure, oleic (5.0 mg/ml), linoleic (5.0 mg/ml), linolenic (3.0 mg/ml) and arachidonic (0.0 mg/ml) fatty acids were prepared in deuterochloroform containing a small amount of butyl hydroxyl toluene (as anti-oxidant) and tetramethylsilane (TMS, as a resonance reference standard). All spectra were collected on a Varian Unity Inova 600 MHz spectrometer using a Nalorac triple resonance probe. Spectra were collected using the modified HSQC sequence as described above (and shown in FIG. 6) with the following pulse sequence parameters. A spectral width of 600 Hz was chosen for the proton dimension and a total of 256 complex points were collected (i.e. np=512). Inline digital filtering was used for the collection of free induction decays using an analog filter with a filter bandwidth of 1000 Hz and an over-sampling bandwidth of 600 Hz. In the indirect dimension, because there was an interest in a very small bandwidth of resonances, the spectral width was chosen to be equivalent to the effective bandwidth of the sech/tan h inversion pulse and covered a range of 700 Hz. The number of increments (ni) could then be reduced allowing collection of the spectra shown in FIGS. 9-11 with ni=64.

Spectra from each of these standards were initially used to determine chemical shifts and once resonances were assigned, they were used to quantify individual unsaturated fatty acids. To verify that chemical shifts and peak intensities were indeed due to different fatty acid species, two test mixtures were made containing oleic (4 & 11.2 mg/ml), linoleic (15 & 30 mg/ml), and linolenic (9 & 12 mg/ml) fatty acids. A maximum receiver gain was determined for the strongest sample and this was used for all standards and samples. Gain corrections factors may be used as described previously (Tesiram Y A. et al., BBA. 2005; 1737:61-68.), but since signals from strong solvent peaks are not detected with this method, it was not necessary to change the receiver gain.

### Biological Samples

Liver extract samples were prepared as follows. Briefly, approximately 250 mg of liver tissue was homogenized in an anoxic environment and extracted with chloroform/methanol (2:1 v/v), concentrated to less than 0.1 ml and reconstituted in deuterated chloroform for NMR analysis. Spectra were collected as described for the standards above. The free induction decay signal data was Fourier transformed and phased using the 2D FT routine in VNMR 6.1C (Varian Inc., Palo Alto) software, saved in double precision and other analyses, including thresholding, projection reconstruction, and calculation of peak areas and concentrations were conducted with Mathematica software (Wolfram, Research In., Illinois). Unknown concentrations were determined using a single standard concentration as reference.

### Chemical Shift Assignment

The absolute chemical shifts of the resonances from pure fatty acids have not been completely determined in this experiment so the chemical shifts reported here are based on the transmitter position, the chosen spectral widths and the resonance frequency of tetramethylsilane (TMS), in addition to comparison with previously published chemical shifts of synthetic olefins (Sandri J., et al. Magn. Reson. Chem. 1997; 35:785-794.). Using a low power continuous decoupling method (Bendall M. R., et al., J. Magn. Reson. 1999; 139:175-80.) the resonance frequency of TMS was determined to be approximately −10 kHz relative to the transmitter frequency in the ¹³C dimension. In the proton dimension, the resonance frequency of TMS was determined to be −1974 Hz downfield of the transmitter frequency (i.e. arbitrary zero position is 3.2 ppm). With the same low power decoupling method the resonance frequency of ¹³C of the vinyl group was determined to be +9640 Hz relative to the decoupler transmitter frequency. Thus, the central point in the in-direct spectrum is approximately 130 ppm, in close correspondence with that reported by Sandri et al. ((Sandri J, et al. Magn. Reson. Chem. 1997; 35: 785-794.). For the purpose of this example, all chemical shifts reported here are based on the arbitrary assignment of the decoupler transmitter frequency set at 130.0 ppm and the chemical shifts of target resonances of the free fatty acid is discussed as the spread of frequencies in Hz around the decoupler transmitter frequency. The proton chemical shifts are of little consequence because the maximum intensity projection of the in-direct dimension is used for analysis. It should also be noted that because pure standards are used as calibration samples, there will be a small shift in frequencies from samples because the compounds from future liver tissue samples are not expected to be pure fatty acids. Rather, the fatty acid moieties are expected to arise from side chains of the glycerol backbone of the phospholipids comprised in cellular membrane and intra- and interstitial fluid of cells.

Generally, differences in chemical shifts are very small compared to the resolution of the spectral dimension and can be affected by differences in shimming quality and the constituents of the sample matrix.

All scientific articles, publications, abstracts, patents and patent applications mentioned herein, including the following, are hereby incorporated by reference in their entirety.

### REFERENCES

1. Iorio E, Mezzanzanica D, Alberti P, Spadaro F, Ramoni C, D'Ascenzo S, Millimaggi D, Pavan A, Dolo V, Canvari S, Podo F. Alterations of choline phospholipid metabolism in ovarian tumor progression. Cancer Res. 2005; 65(20): 9369-9376.
2. Whitehead T L, Monzavi-Karbassi B, Jousheghany F, Artaud C, Elbein A, Kieber-Emmons T. ¹H-NMR metabolic markers of malignancy correlate with spontaneous metastases in a murine mammary tumor-model. Int. J. Oncology. 2005; 27:257-263.
3. Katz-Bull R, Seger D, Rivenson-Segal, Rushkin E, Degani H. Metabolic markers of breast cancer: Enhanced cholinemetabolism and reduced choline-ether-phospholipid synthesis. Cancer Res. 2002; 62: 1966-1970.
4. Klein J, Gonzalez, Köppen A, Löffelholz K. Free choline and choline metabolites in rat brain and body fluids: Sensitive determination and implications for choline supply to the brain. Neurochem. Int. 1993; 22(3): 293-300.
5. Kobliakov V A, Somova O G, Kondalenko V F, Ostashkina W M, Kandyba A G, Duboyaya T K, Dyatlovitskaya E V. Differences in lipid composition and proliferative activity of rat hepatoma-27 depending on the target organ. Biochemistry. 2001; 66: 603-607.
6. Moore S, Knudsen B, True L D, Hawley S, Etzioni R, Wade C, Gifford D, Coleman I, Nelson P S. Loss of stearoyl-CoA desaturase expression is a frequent event in prostrate carcinoma. In. J. Cancer. 2005; 114: 563-571.
7. Horrobin D F. Fatty acid metabolism in health and disease—the role of ω6-desaturase. Am. J. Clin. Nutr. 1993; 57: 5732-5737.
8. Lane J, Mansel R E, Jiang W G. Expression of human ω6-desaturase is associated with aggressiveness of human breast cancer. Int. J. Mol. Med. 2003; 12: 253-257.
9. Tesiram Y A. Sanders D., Towner R A. Application of proton NMR spectroscopy in the study of lipid metabolites in a rat hepatocarcinogenesis model. BBA. 2005; 1737: 61-68.
10. Kay L E, Keifer P, Saarinen T. Pure absorption gradient enhanced single quantum correlation spectroscopy with improved sensitivity. J. Am. Chem. Soc. 1992; 114: 10663-10665.
11. Bendall M R, Skinner T E. J pulses for multiplet-selective NMR. J. Magn. Reson. 1999; 141: 261-70.
12. Silver M S, Joseph R I, Hoult D I. Selective spin inversion in nuclear magnetic resonance and coherent optics through an exact solution of the Bloch-Riccati equation, Phys. Rev. A. 1985; 31: 2753-2755.
13. Bendall M R, Skinner T E. Comparison and use of vector and quantum representations of J-coupled spin evolution in an IS spin system during RF irradiation of one spin. J. Magn. Reson. 2005; 143: 329-351.
14. Tesiram Y A, Bendall M R. Universal equations for linear adiabatic pulses and characterization of partial adiabaticity. J. Magn. Reson. 2002; 156: 26-40.
15. Sandri J, Soto T, Gras J-L, Viala J. Chemical shifts of ethylenic carbons in polyunsaturated fatty acids and related compounds. Magn. Reson. Chem. 1997; 35: 785-794.
16. Bendall M R, Skinner T E. Novel methods for characterizing a decoupler channel using “undetectable” quantum coherences. J. Magn. Reson. 1999; 139: 175-80.
17. Ntambi J M, Miyazaki M. Regulation of stearoyl-CoA desaturase and role in metabolism. Pro. Lipid. Res. 2004; 43: 91-104.
18. B. W. Stewart, P. Kleihues, WORLD HEALTH REPORT, World Health Organisation, IARC Press, Lyon, 2003.
19. E. Farber, Alcohol and other chemicals in the development of hepatocellular carcinoma, Clin. Lab. Med. 16 (1966) 377.
20. D. Nakae, Endogenous liver carcinogenesis in the rat, Pathol. Int. 49 (1999) 1028-1042.
21. P. E. Sijens, M. V. Knopp, A. Brunetti, K. Wicklow, B. Alfano, P. Bachert, J. A. Sanders, A. E. Stillman, H. Kett, R. Sauter, ¹H-MRS in patients with metastatic brain tumors: a multicenter study, J. Magn. Reson. Imaging 33 (1995) 818-826.
22. H. Kugel, W. Heindel, R. I. Ernestus, J. Bunke, R. du Mesnil, G. Friedmann, Human brain tumors: spectral patterns detected with localized ¹H MRS, Radiology 183 (1992) 701-709.
23. D. Ott, J. Hennig, T. Ernst, Human brain tumors: assessment with in vivo proton MR spectroscopy, Radiology 186 (1993) 745-752.
24. G. Hagberg, A. P. Burlina, I. Mecder, W. Roser, E. W. Radue, J. Seelig, In vivo proton MRS of human gliomas: definition of metabolic coordinates for multi-dimensional classification, Magn. Reson. Med. 34 (1995) 242-252.
25. T. R. McKnight, M. H. von dem Bussche, D. B. Vigneron, Y. Lu, M. S. Berger, M. W. McDermott, W. P. Dillon, E. E. Graves, A. Pirzkall, S. J. Nelson, Histopathological validation of a three-dimensional magnetic resonance spectroscopy index as a predictor of tumor presence, J. Neurosurg. 97 (2002) 794-802.
26. D. K. Yeung, W. T. Yang, G. M. Tse, Breast cancer: in vivo proton MR spectroscopy in the characterization of histopathologic subtypes and preliminary observations in axillary node metastases, Radiology 225 (2002) 190-197.
27. J.-K. Kim, S.-H. Park, H. M. Lee, Y.-H. Lee, N.-K. Sung, In vivo ¹H-MRS evaluation of malignant and benign breast diseases, The Breast 12 (2003) 179-182.
28. M. Hasumi, K. Suzuki, N. Oya, K. Ito, K. Kurokawa, Y. Fukabori, H. Yamanaka, MR spectroscopy as a reliable diagnostic tool for localization of prostate cancer, Anti-cancer Res. 22 (2002) 1205-1208.
29. J. W. Kwek, C. H. Thng, MR imaging and MR spectroscopy of adenocarcinoma of the prostate, Ann. Acad. Med. Singap. 32 (2003) 500-506.
30. R. Longo, C. Ricci, F. Masutti, R. Vidimari, L. S. Croce, L. Bercich, C. Tiribelli, L. Dalla Palma, Fatty infiltration of the liver. Quantification by ¹H localized magnetic resonance spectroscopy and comparison with computed tomography, Invest. Radiol. 28 (1993) 297-302.
31. J. Folch, M. Lees, G. H. Sloane Stanley, A simple method for the isolation and purification of total lipids from animal tissues, J. Biol. Chem. 226 (1957) 497-509.
32. C. M. Smuts, H. F. H. Weich, J. Weight, M. Faber, M. Kruger, C. J. Lombard, A. J. S. Benade´, Free cholesterol concentrations in the high density lipoprotein subfraction-3 as a risk indicator in patients with angiographically documented coronary artery disease, Coron. Artery Dis. (1994) 331-338.
33. J. L. Griffin, K. K. Lehtima\ddot{}ki, P. K. Valonen, O. H. J. Gröhn, M. I. Kettunen, S. Yla\ddot{}-Herttuala, A. Pitkaönen, J. K. Nicolson, R. Kauppinen, Assignment of ¹H nuclear magnetic resonance visible polyunsaturated fatty acids in BT4C gliomas undergoing ganciclovir-thymidine kinase gene therapy-induced programmed cell death, Can. Res. 63 (2003) 3195-3201.
34. A. Savitzky, M. J. E. Golay, Smoothing and differentiation of data by simplified least squares procedures, Anal. Chem. 36 (1964) 1627-1629.
35. S. Abel, C. M. Smuts, C. de Villiers, W. C. Gelderblom, Changes in essential fatty acid patterns associated with normal liver regeneration and the progression of hepatocyte nodules in rat hepatocarcinogenesis, Carcinogenesis 22 (2001) 795-804.
36. W. C. A. Gelderblom, W. Moritz, S. Swanevelder, C. M. Smuts, S. Abel, Lipids and D6-desaturase activity alterations in rat liver microsomal membranes induced by fumonisin B1, Lipids 37 (2002) 869-877.
37. A. Ferretti, A. Knijn, E. Iorio, S. Pulciani, M. Giambenedetti, A. Molinari, S. Meschini, A. Stringaro, A. Calcabrini, I. Freitus, R. Strom, G. Arancia, F. Podo, Biophysical and structural characterization of ¹H NMR detectable mobile lipid domains in NIH-3T3 fibroblasts, Biophys. Biochim. Acta 1438 (1999) 329.
40. W. A. Cooper, W. A. Bartier, D. C. Rideout, Delikatny, ¹H NMR visible lipids are induced by phosphonium salts and 5-fluorouracil in human breast cancer cells, Magn. Reson. Med. 45 (2001) 1001-1010.
41. E. J. Delikatny, W. A. Cooper, S. Brammah, N. Sathasivam, D. C. Rideout, Nuclear magnetic resonance visible lipids induced by cationic lipophilic chemotherapeutic agents are accompanied by increased lipid droplet formation and damaged mitochondria, Cancer Res. 62 (2002) 1394-1400.
42. L. M. Foley, R. A. Towner, D. M. Painter, In-vivo image guided ¹H NMR spectroscopy of the serial development of hepatocarcinogenesis in an experimental rat model, Biophys. Biochim. Acta 1526 (2001) 230-236.
43. R. A. Towner, L. M. Foley, D. M. Painter, Hepatocarcinogenesis tumour grading correlated with in vivo image-guided ¹H NMR spectroscopy in a rat model, Toxicol. Applied. Pharmacol. (2005).
44. L. Tesitore, B. Marengo, D. E. Vance, M. Papotti, A. Mussa, M. Grazia Daidone, A. Costa, Expression of phosphatidylethanolamine N-methyltransferase in human hepatocellular carcinomas, Oncology 65 (2003) 152-158.
45. Y. C. Kim, J. M. Ntambi, Regulation of stearoyl-CoA desaturase genes: role in cellular metabolism and preadipocyte differentiation, Biochem. Biophys. Res. Commun. 266 (1999) 1-4.
46. M. Miyazaki, J. M. Ntambi, Role of stearoyl-CoA desaturase in lipid metabolism, Prostaglandins Leukot. Essent. Fat. Acids 68 (2003) 113-121.
47. J. M. Ntambi, M. Miyazaki, Recent insights into stearoyl-CoA desaturase-1, Curr. Opin. Lipodol. 14 (2003) 255-261.
48. Brown et al. (PNAS 79: 3523-3526 (1982).
49. Mansfield (Magn. Reson. Med., 1(3): 370-386 (1984).

With the above disclosed embodiments, it should be apparent that the present invention is directed to a general method of determining metabolite differences in an individual that has a biological condition. Thus, the present invention should not to be limited by the above disclosed specific embodiments but should rather be embraced by the below claims.

We claim:

1. A method for detecting an alteration of a metabolite marker in a sample, the method comprising:
subjecting the sample to nuclear magnetic resonance spectroscopy or magnetic resonance imaging to determine the concentration of the metabolite marker in the sample; and
comparing the concentration of the metabolite marker in the sample to a concentration from a normal sample;
wherein a difference between the concentration of metabolite marker in the sample and the normal sample indicates an alteration in the metabolite marker in the sample.

2. The method of claim 1, wherein the sample is subjected to nuclear magnetic resonance spectroscopy selected from the group consisting HSQC, HMQC, HMQC-NOESY, HMQC-TOCSY, HMBC, and INEPT.

3. The method of claim 1, wherein the sample is subjected to magnetic resonance imaging and the concentration of metabolite marker is determined by chemical shift imaging.

4. The method of claim 1, wherein the metabolite marker is selected from the group consisting of a lipid, a fatty acid, a protein, a nucleic acid, a vitamin, a peptide, a sugar, an amino acid, a phospholipid, and a steroid.

5. The method of claim 1, wherein the metabolite markers is a fatty acid selected from the group consisting of oleic acid, linoleic acid, linolenic acid, and arachidonic acid.

6. A method of detecting a pathological condition in an individual, the method comprising:
subjecting the sample to nuclear magnetic resonance spectroscopy or magnetic resonance imaging to determine the concentration of a metabolite marker in the sample that is known to change in concentration when the pathological condition is present; and
comparing the concentration of the metabolite marker in the sample to a concentration from a normal sample when the biological condition is present to when the biological condition is not present;
wherein a difference between the concentration of metabolite marker in the sample and the normal sample indicates the presence of the pathological condition in the individual.

7. The method of claim 6, wherein the biological condition is selected from the group consisting of cardiovascular disease, obesity, diabetes, neurological disease, and cancer.

8. The method of claim 6, wherein the sample is subjected to nuclear magnetic resonance spectroscopy and the concentration of metabolite marker is determined using HSQC, HMQC, HMQC-NOESY, HMQC-TOCSY, HMBC, or INEPT.

9. The method of claim 6, wherein the sample is subjected to nuclear magnetic resonance imaging and the concentration of metabolite marker is determined by chemical shift imaging.

10. The method of claim 6 wherein the metabolite marker is selected from the group consisting of a fatty acid, a protein, a nucleic acid, a vitamin, a peptide, a sugar, an amino acid, a phospholipid, and a steroid.

11. The method of claim 6, wherein the metabolite marker is a fatty acid selected from the group consisting of oleic acid, linoleic acid, linolenic acid, and arachidonic acid.

12. The method of claim 6, wherein the pathological condition is cancer.

13. The method of claim 6, wherein the method uses HSQC to determine the concentration of a fatty acid metabolite in the sample and the pathological condition is liver cancer.

14. A method of increasing resolution of one or more chemical species in an NMR spectrum of a sample comprising:
applying one or more tailored radiofrequency pulses to the sample to determine directly or indirectly the one or more chemical species present in a class of compounds by limiting an NMR signal generated to one or more small regions of the NMR spectrum, and
detecting a set of resonances in the one or more small regions of the NMR spectrum,
wherein the resolution of one or more chemical species in the NMR spectrum is increased.

15. The method of claim 14, wherein the class of compounds are one or more members selected from the group consisting of lipids, fatty acids, proteins, nucleic acids, vitamins, peptides, sugars, amino acids, phospholipids, and steroids.

16. The method of claim 14, wherein the one or more tailored radiofrequency pulses comprise a hermite J pulse.

17. The method of claim 14, wherein the one or more chemical species are determined by one or more of HSQC, HMQC, HMQC-NOESY, HMQC-TOCSY, HMBC, TOCSY, or INEPT.
